(19) United States
(12) Patent Application Publication
(10) Pub. No.: US 2014/0114436 A1
(43) Pub. Date: Apr. 24, 2014

(54) MEDICAL DEVICES AND METHODS FOR IMPROVING THE BIOCOMPATIBILITY OF MEDICAL DEVICES

(71) Applicants: Patrick A. Tresco, Sandy, UT (US); Michael J. Bridge, Salt Lake City, UT (US)

(72) Inventors: Patrick A. Tresco, Sandy, UT (US); Michael J. Bridge, Salt Lake City, UT (US)

(21) Appl. No.: 14/000,008

(22) PCT Filed: Feb. 17, 2012
(86) PCT No.: PCT/US12/25589

§ 371 Date: Dec. 23, 2013

Related U.S. Application Data
(60) Provisional application No. 61/443,863, filed on Feb. 17, 2011.

(51) Int. Cl. A61L 27/52

(52) U.S. Cl. CPC A61L 27/52
USPC 623/23.76; 427/2.24; 156/60

(57) ABSTRACT

A medical device configured to be at least partially implanted within a host. The medical device includes an outer surface at least a portion of which is impermeable to cells but is permeable to molecules secreted by cells, the outer surface separating a space inside the medical device from a surrounding tissue of the host. The medical device also includes a diffusion sink positioned within the space and configured to cause molecules that are secreted into the tissue by immune cells during a foreign body response (FBR) to diffuse, through random motion, through the portion and into the diffusion sink.

### CROSS-REFERENCE TO RELATED APPLICATIONS

This application claims the benefit of U.S. Provisional Application No. 61/443,863, filed Feb. 17, 2011, the content of which is incorporated herein by reference in its entirety.

### STATEMENT REGARDING FEDERAL FUNDING

This invention was made with government support under NS046770 and N66001-06-C-8005 awarded by National Institutes of Health and DOD/DARPA. The government has certain rights in this invention.

### INTRODUCTION

The response of a host's tissues to the presence of indwelling or implanted medical devices and prosthetics, which is referred to herein as the foreign body response (FBR), often compromises the intended function of the medical device, and has the potential to cause device failure. Generally, implantable Class II and Class III medical devices are solid and consist of one or more materials that are impermeable to water and the molecules dissolved or carried in the water. The foreign body response (FBR) to such implantable medical devices begins immediately after implantation and continues for as long as the device remains in the body.

Irrespective of the type of medical device, or the location the device has been implanted, the FBR described in the scientific literature is very similar. The FBR occurs within the tissues surrounding and adjacent to the outer surface of the implanted or indwelling medical device, and is characterized by persistent macrophage activation, persistent inflammatory sequelae (such as pain, swelling and edema), cellular necrosis, and fibrous encapsulation (scarring). It has been observed that, during the FBR, macrophages and other immune cells (including but not limited to neutrophils during the early phase of the FBR, and immune effector cells during later stages of the FBR) secrete water soluble molecules, such as proteolytic enzymes, proinflammatory and cytotoxic cytokines (e.g., MCP-1, IL-1B, IL-6, and TNF-α, reactive oxygen intermediates, leucotriens, and NO, among others. Persistent or chronic exposure of tissues to these secreted molecules is not a normal condition, and generally is only observed following traumatic injury, stroke, near degenerative disease foci or surrounding infectious agents. Available scientific evidence supports the notion that these secreted molecules are responsible for the FBR of the tissues surrounding implanted or indwelling medical devices, which ultimately causes device migration, decreased device performance, and/or complete device failure.

Current attempts to reduce the FBR to implanted or indwelling medical devices, or to reduce the concomitant effects of the FBR, include the use of medical devices with significant surface modifications, and the use anti-inflammatory drugs. These methods have met with limited success.

### SUMMARY

This disclosure provides medical devices configured to be at least partially implanted within a host and having improved biocompatibility over existing medical devices. The medical devices include an outer surface at least a portion of which is impermeable to cells but is permeable to water and molecules secreted by cells, the outer surface separating a space inside the medical device from a surrounding tissue of the host. The medical devices also include a diffusion sink positioned within the space and configured to cause molecules that are secreted into the tissue by immune cells during a foreign body response (FBR) to diffuse, through random motion, through the portion of the outer surface and into the diffusion sink.

The diffusion sink may include a semi-permeable material, where the portion of the outer surface includes an outer surface of the semi-permeable material. For example, the semi-permeable material may include a hydrogel, a film or a membrane. The semi-permeable material may be at least about 50 μm thick, such as at least about 100 μm thick, at least about 200 μm thick, at least about 400 μm thick, at least about 600 μm thick, or at least about 1 mm thick. The semi-permeable material may include a plurality of pores sized to accommodate the passage of molecules of selected sizes and to preclude the passage of cells. These pore sizes may be selected to accommodate passage of selected molecules, including but not limited to proteins, reactive oxygen intermediates, proinflammatory and cytotoxic cytokines, and/or other molecules produced by macrophages and/or other immune cells during the foreign body response. The pore sizes may be selected and/or optimized to preclude the passage of cells, including but not limited to macrophages and other immune cells, tissue cells, and the like. The volume of the diffusion sink may be selected to be at least about 25% the volume of the adjacent tissue within which it is implanted.

In some embodiments, the diffusion sink may include a reservoir (e.g., a basin, container, compartment, sink, well, vessel, passageway, tube, pipe, duct, chimney, flue, etc.) that is separated from the surrounding tissue of the host by the semi-permeable material, and is in fluid communication with the permeable portion of the outer surface due to the permeability of the semi-permeable material to water. The reservoir may be at least partially defined by at least one wall formed of an impermeable material (i.e., a material that is impermeable to water). The reservoir may be at least partially filled with the semi-permeable material. In some embodiments, the semi-permeable material may be a first semi-permeable material including a first plurality of pores having a first average pore size, and the reservoir may be at least partially filled with a second semi-permeable material including a second plurality of pores having a second average pore size that is larger than the first average pore size.

Some of the medical devices disclosed herein may include a class II or class III medical device having an outer wall at least partially formed of a semi-permeable material. Alternatively or additionally, some medical devices may include a class II or class III medical device having an outer wall formed of a substantially impermeable material (i.e., a material that is impermeable to water), where the semi-permeable material has been applied to at least a portion of the outer wall of the class II or class III medical device to form the implantable medical device.

In some embodiments, the medical device may include a plurality of surface members, each surface member extending outwardly to an end, wherein the semi-permeable material forms a layer that engages the ends of the surface members such as, for example, to define a plurality of reservoirs.

This disclosure also provides methods of improving the biocompatibility of a medical device, which include providing a class II or class III medical device having an outer wall, and applying a semi-permeable material to the outer wall to form a diffusion sink having an outer surface that is impermeable to cells but is permeable to molecules secreted by cells. The diffusion sink is configured to cause molecules that are secreted by cells adjacent to the outer surface during a foreign body response (FBR) to diffuse, through random motion, through the portion and into the diffusion sink. The outer wall of the class II or class III medical device may be substantially impermeable to water. The semi-permeable material may include at least one of a hydrogel, a porous film, a membrane or a porous media that permits the diffusive transport of molecules into its bulk dimensions, and may be applied to a thickness of at least about 50 μm, such as at least about 100, at least about 200 μm, at least about 400 μm, at least about 600 μm, or at least about 1 mm. The diffusion sink may include a reservoir that is in fluid communication with the outer surface due to the permeability of the semi-permeable material to water. The class II or class Ill medical device may include a plurality of surface members, each surface member extending outwardly to an end, wherein the semi-permeable material forms a layer that engages the ends of the surface members.

### BRIEF DESCRIPTION OF THE DRAWINGS

FIG. 1 is a schematic showing exemplary designs for the medical devices disclosed herein.

FIG. 2 is a schematic showing additional exemplary designs for the medical devices disclosed herein.

FIG. 3 is a graph showing a plot of the theoretical concentration of cytokine TNF-α, at steady state equilibrium, as a function of position relative to regions surrounding the flat interface between the semi-permeable exterior surface of a diffusion sink and the tissue of a host.

FIG. 4 is a schematic showing the results of a series of modeling studies predicting the theoretical concentrations of cytokine TNF-α, at steady state equilibrium, in regions surrounding the interfaces between the exterior surfaces of various flat medical devices and the tissues of hosts within which each medical device is implanted.

FIG. 5 is a graph showing a series of plots, each showing the theoretical concentration of cytokine TNF-α, at steady state equilibrium, as a function of the distance relative to the interface between the exterior surface of a flat medical device and the tissue of a host within which the medical device is implanted.

FIG. 6 is a graph showing plots of the theoretical equilibrium sink ratio (i.e., the ratio of cytokine TNF-α in a diffusion sink to cytokine TNF-α in the surrounding tissue of the host at steady state equilibrium) and the sink efficiency (i.e., the percentage of total cytokine TNF-α in the diffusion sink at steady state equilibrium) as functions of the width of the diffusion sink for flat medical devices.

FIG. 7 is a schematic showing the results of a series of modeling studies predicting the theoretical concentrations of cytokine TNF-α, at steady state equilibrium, in regions surrounding the interfaces between the exterior surfaces of various cylindrical medical devices and the tissues of hosts within which each medical device is implanted.

FIG. 8 is a graph showing a series of plots, each showing the theoretical concentration of cytokine TNF-α, at steady state equilibrium, as a function of the distance relative to the interface between the exterior surface of a cylindrical medical device and the tissue of a host within which the medical device is implanted.

FIG. 9 is a graph showing plots of the theoretical equilibrium sink ratio and the sink efficiency as functions of the width of the diffusion sink for various cylindrical medical devices.

FIG. 10 is a series of graphs showing a series of plots, each plot showing the theoretical concentration of cytokine TNF-α, at steady state equilibrium, as a function of the distance relative to the interface between the exterior surface of a cylindrical medical device and the tissue of a host within which the medical device is implanted.

FIG. 11 is a graph showing plots of the theoretical equilibrium sink ratio and the sink efficiency as functions of the width of the diffusion sink for various cylindrical medical devices.

FIG. 12 is a schematic showing cylindrical implantable devices having (top) a diffusion sink with a semi-permeable exterior layer and an open reservoir, and (bottom) a diffusion sink with a semi-permeable exterior an occluded reservoir.

FIG. 13 is series of fluorescence microcopy images comparing distributions of (A and C) ED-1+ macrophages and (B and D) GFAP+ astrocytes found in tissues surrounding devices with a diffusion sink having (A and B) an open reservoir (i.e., “Open Sink”) or (C and D) an occluded reservoir (i.e., “Occluded Sink”) 12 weeks after each device was implanted in a host tissue.

FIG. 14 is a pair fluorescence microscopy images comparing distributions of GFAP+astrocytes around the implantation region of devices with a diffusion sink having (A) an open reservoir (i.e., “Open Sink Device”) or (B) an occluded reservoir (i.e., “Occluded Sink Device”) 12 weeks after each device was implanted in a host tissue.

FIG. 15 is a pair fluorescence microscopy images comparing the density of cells (stained green by Phalloidin and blue by DAPI) surrounding (A) uncoated microelectrodes and (B) electrodes coated with an alginate hydrogel.

FIG. 16 is an image showing (left) an uncoated planar silicon microelectrode array, and (right) an alginate coated microelectrode array following hydration.

FIG. 17 is a pair of fluorescence microscopy images showing ED-1+ macrophages in tissues adjacent to the implantation tracts of (A) uncoated microelectrodes and (B) alginate coated microelectrodes 12 weeks after the devices were implanted in the tissues.

FIG. 18 is a graph comparing plots of the fluorescence intensity of ED-1 stained tissues as a function of the distance of the tissue from the device-tissue interface 12 weeks after implantation of uncoated and alginate-coated devices.

FIG. 19 is a pair of fluorescence microscopy images showing GFAP+ astrocytes in tissues adjacent to the implantation tracts of (A) uncoated microelectrodes and (B) alginate coated microelectrodes 12 weeks after the devices were implanted in the tissues.

FIG. 20 is a graph comparing plots of the fluorescence intensity of GFAP stained tissues as a function of the distance of the tissue from the device-tissue interface 12 weeks after implantation of uncoated and alginate-coated devices.

FIG. 21 is a pair of fluorescence microscopy images showing NeuN+ neuronal cells in tissues adjacent to the implantation tracts of (A) uncoated microelectrodes and (B) alginate coated microelectrodes 12 weeks after the devices were implanted in the tissues.

FIG. 22 is a graph comparing plots of the percentage of normal neuronal density as a function of the distance of the tissue from the device-tissue interface 12 weeks after implantation of uncoated and alginate-coated devices.

FIG. 23 is a bar chart showing the integrated area-under-the-curve (AUC) analysis for ED1, GFAP, and NeuN positive nuclei from 0-50 μm distance adjacent to the electrode tissue-tissue interface.

### DETAILED DESCRIPTION

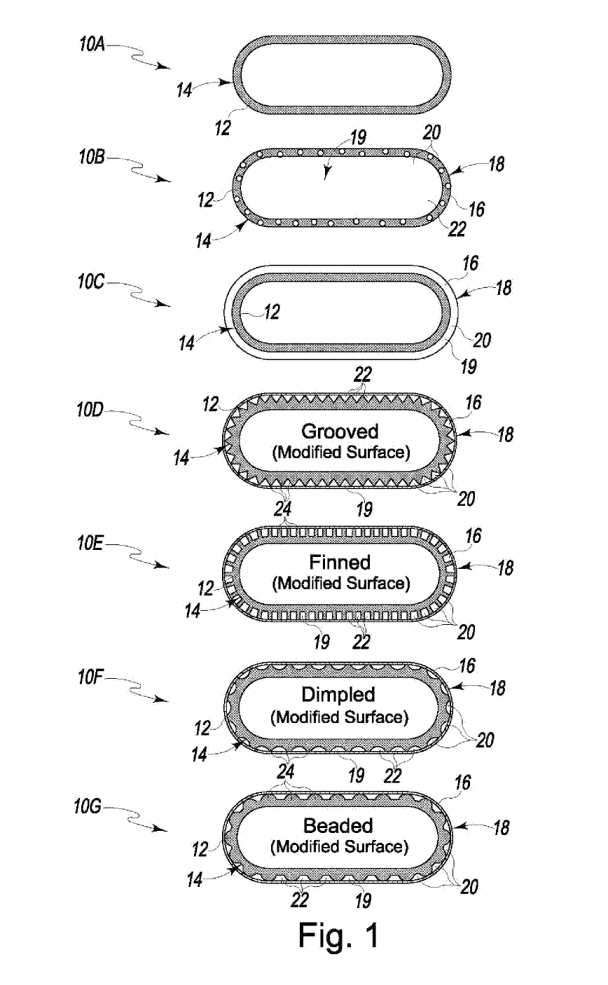

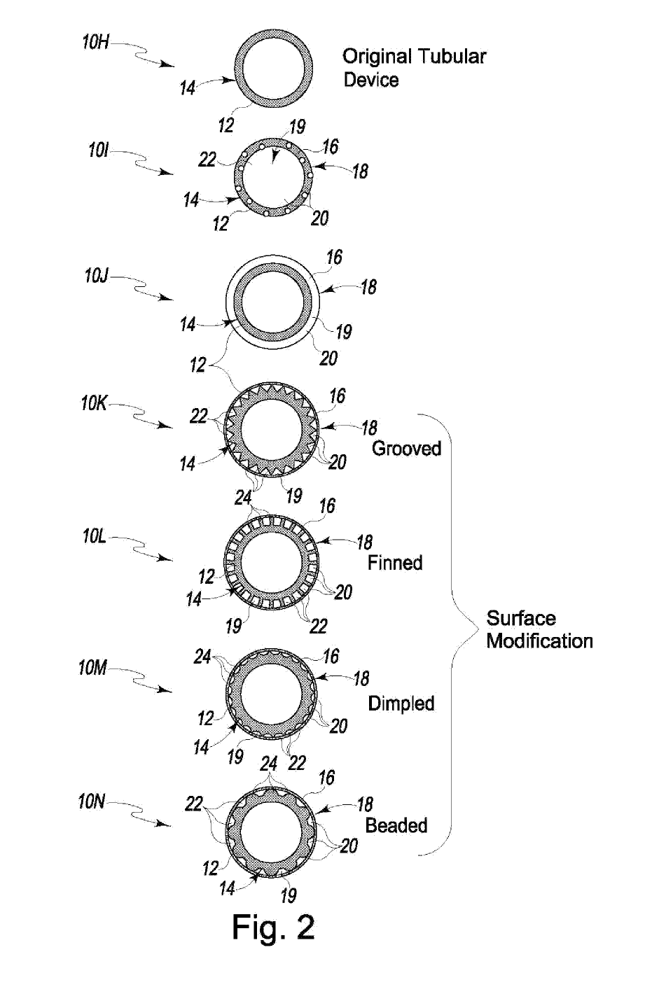

FIGS. 1 and 2 show non-limiting examples of implantable medical devices 10. Generally, existing implantable medical devices, such as those depicted as devices 10A and 10H, may include a device housing 12 having an outer surface 14. The housing 12 may be formed of a polymeric material that is substantially impermeable to water and/or molecules soluble in water. The outer surface 14 (which also may define the device-tissue interface when implanted in the tissue of a host) thus also may be impermeable to water and/or molecules soluble in water.

In the United States, implantable medical devices are classified as either class II or class III medical devices under 21 C.F.R. §860, and are subject to special controls. Examples of Class II and Class III medical devices may include, but are not limited to: cardiovascular prosthetic devices, such as vascular clips (II), implantable pacemaker pulse generators (III), pacemaker lead adaptors (II), pacemaker electrodes (III), and carotid sinus nerve stimulators (III) among others; ear, nose, and throat devices such as implantable hearing prostheses (II) (i.e., partial and total ossicular replacement prostheses), endolymphatic shunts (II), endolymphatic shunt tubes with valves (II), tympanostomy tubes (II), tympanostomy tubes with semi-permeable membranes (II), and middle ear molds (II), among others; gastroenterology and urology devices, such as penile inflatable implants (III), penile rigidity implants (II), testicular prostheses (III), implanted electrical urinary continence devices (III), and implanted mechanical/hydraulic urinary continence devices (III), among others; general and plastic surgery devices, such as surgical meshes (II), silicone inflatable breast prostheses (III), silicone gel-filled breast prostheses (III), chin prostheses (II), ear prostheses (II), esophageal prostheses (II), nose prostheses (II), tracheal prostheses (II), implantable clips (II), and nonabsorbable sutures (II), among others; general hospital devices, such as implanted percutaneous long-term intravascular catheters (II); neurological devices, such as nerve cuffs (II), central nervous system fluid shunts (II), implanted cerebellar stimulators (III), implanted diaphragmatic/phrenic nerve stimulators (III), implanted intracerebral/subcortical stimulators for pain relief (III), implanted spinal cord stimulators for bladder evacuation (III), implanted neuromuscular stimulators (III), implanted peripheral nerve stimulators for pain relief (II), and implanted spinal cord stimulators for pain relief (II), among others; obstetrical and gynecological devices, such as fallopian tube prostheses (II), among others; ophthalmic devices, such as eye sphere implants (II), and aqueous shunts (II), among others; implantable recording/stimulating electrodes used in brain machine interfaces and advanced prosthetic technologies (unclassified experimental device); implantable glucose sensors (unclassified experimental device); implantable drug delivery pumps (unclassified experimental device); and implanted extended release drug delivery capsules.

The implantation of existing class II and class III medical devices may be followed by a typical foreign body response (FBR), which surrounds the device and is characterized by persistent macrophage activation near the device surface, persistent inflammation, cellular necrosis and fibrous encapsulation (scarring). The FBR may result in a decrease in device performance. A consistent feature of the FBR may be persistent inflammation resulting from macrophage activation, which serves as a potent source of proinflammatory and cytotoxic cytokines. Molecules secreted by immune cells in the adjacent tissues during the FBR may become concentrated at the device-tissue interface and/or attached to the surface of the device. Persistent or chronic exposure of adjacent tissues to these secreted molecules may damage the tissues and/or the device itself. This problem may be ameliorated either by re-designing existing medical devices to have diffusion sinks, or by applying a diffusion sink to the existing medical device.

This disclosure provides implantable medical devices having an outer surface at least a portion of which is permeable to water and molecules soluble in water, such as macrophage secreted molecules, and having diffusion sinks that are configured to transport such molecules into the device and away from the surrounding tissue to reduce the effects of the FBR and improve the biocompatibility of the device. This disclosure also provides methods of improving the biocompatibility of an implantable medical device that includes applying a semi-permeable material to the outer wall to form a diffusion sink. The medical devices disclosed herein may be designed to include diffusion sinks, and/or may be formed by applying a diffusion sink to an existing medical device that otherwise lacks a diffusion sink (i.e., retrofitting an existing medical device).

Medical devices 10B-10G and 10L-10N may represent medical devices designed to include diffusions sinks. Each of these medical devices may include a semi-permeable material 16 that defines a semi-permeable outer surface 18 separating a space 19 within the medical device from the space outside the medical device, and a diffusion sink 20 positioned within the space. The diffusion sink may include the semi-permeable material 16 and/or one or more reservoirs 22 in fluid communication with the outer surface 18 due to the permeability of the semi-permeable material. In some embodiments, such as those depicted as devices 10B and 10I, the main housing 12 of the medical device may be formed of the semi-permeable material 16, and the outer surface 14 of the housing may be the semi-permeable outer surface 18. In some embodiments, such as those depicted as devices 10C-10G and 10J-10N, the housing 12 of the medical device may be formed of a substantially impermeable material, and may be at least partially surrounded by the semi-permeable material 16, such as by a layer of the semi-permeable material. The surface 14 of the main housing 12 may be flat, or may have one or more surface members 24 each of which extends outwardly to an end, thereby causing the housing to have a grooved, finned, dimpled, beaded, or corrugated structure, among other structures, depending on the function of the medical device. The semi-permeable material may be attached to the ends of the surface members.

While medical device 10C-10G and 10J-10N may represent devices specifically designed to include diffusion sinks, they also may represent existing medical devices that have been retrofit to include diffusions sinks. For example, to form the medical devices depicted as devices 10C and 10J, a layer of semi-permeable material 16 may be applied to the outer surfaces 14 of devices 10A or 10H, respectively. Similarly, to form the devices depicted as devices 10D-10G or 10K-10N, a diffusion sink reservoir jacket may be separately manufactured for later application to the outer surfaces 14 of devices 10A or 10H, respectively. These diffusion sink reservoir jackets may include the semi-permeable material 16 attached to one or more scaffolding members 24 that may be attached to the outer surface of the existing medical device. The scaffolding members thus may form the surface members 24. In such embodiments, the housing 12 of the medical device may be formed of a substantially impermeable material, and may be at least partially surrounded by the semi-permeable material 16, such as by a layer of the semi-permeable material.

The semi-permeable material 16 may be formed of any porous material or media that permits diffusive transport of molecules into and/or through its volume, and thus may include a plurality of pores sized to accommodate the passage of molecules of selected sizes and to preclude the passage of cells. For example, the semi-permeable material may include one or more hydrogels (i.e., synthetic and naturally occurring polymers and copolymers with an abundance of hydrophilic groups, including but not limited to polymers and copolymers comprising acrylate, agarose, alginate, hyaluronan, polyvinyl alcohol, sodium polyacrylate, and many others), porous films, and/or porous membranes. The pore sizes may be selected to accommodate passage of selected molecules, including but not limited to proteins, reactive oxygen intermediates, proinflammatory and cytotoxic cytokines, and/or other molecules produced by macrophages and/or other immune cells during the foreign body response. The pore sizes may be selected and/or optimized to preclude the passage of cells, including but not limited to macrophages and other immune cells, tissue cells, and the like. The semi-permeable material 16 may have a bulk dimension that defines a volume, and may be at least about 50 μm thick, such as at least about 100 μm thick, at least about 200 μm thick, at least about 400 μm thick, at least about 600 μm thick, or at least about 1 mm thick.

The semi-permeable outer surface 18 may be formed of the same material as the semi-permeable material 16, and as such, may have the same permeability as the semi-permeable material. Specifically, the semi-permeable outer surface may be impermeable to cells but permeable to water and/or molecules soluble in water. The outer surface 18 also may define the device-tissue interface when implanted in the tissue of a host.

The diffusion sink 20 may include the semi-permeable material 16 and/or one or more reservoirs 22 that is in fluid communication with the outer surface 18 due to the permeability of the semi-permeable material. In embodiments that do not include a separate reservoir, the volume of the diffusion sink may be the volume of the semi-permeable material. In embodiments that do have a reservoir in fluid communication with the semi-permeable material, the volume of the diffusion sink may be the sum of the volumes of the semi-permeable material and the reservoir. The volume of the diffusion sink may be selected to be at least about 10% to about 60% of the volume of the adjacent tissue, e.g., 10% the volume of the adjacent tissue within which it is implanted, such as, for example, at least about 15%, at least about 20%, at least about 25%, at least about 30%, at least about 40%, at least about 50%, or at least about 60% of the volume of the adjacent tissue.

The reservoir 22 may be a basin, container, compartment, sink, well, vessel, passageway, tube, pipe, duct, chimney, flue, or any other suitable structure that is separated from the surrounding tissue of the host by the semi-permeable material, and is in fluid communication with the permeable portion of the outer surface due to the permeability of the semi-permeable material to water. Reservoirs may be at least partially defined by at least one wall formed of an impermeable material 12 (i.e., a material that is impermeable to water). In some embodiments, a reservoir may be at least partially filled with the semi-permeable material. In some embodiments, the semi-permeable material may be a first semi-permeable material including a first plurality of pores having a first average pore size, and the reservoir may be at least partially filled with a second semi-permeable material including a second plurality of pores having a second average pore size that is larger than the first average pore size.

Surface members 24 which, as discussed above, may be integral with the housing 12 or may be separate manufactured and later attached to housing 12 as a scaffolding, each may extend outwardly to an end. The semi-permeable material may then form a layer that engages the ends of the surface members such as, for example, to define reservoirs 22.

The diffusion sinks of the medical devices disclosed herein may draw macrophage secreted factors away from adjacent tissue regions. The efficiency of the diffusion sink to do so may depend on the volume of the sink reservoir relative to that of adjacent tissue and the effective diffusivity of the sink which should be significantly greater than that of the adjacent tissue. Cytokines have relatively short active lives. Following their secretion into extracellular spaces, their tertiary and quaternary molecular structures may become susceptible to denaturation, which may deactivate their signaling functions. In this manner, their range of action is naturally curtailed in vivo which limits the extent of bystander effects in tissue adjacent to a region of injury. Cytokine factors drawn into a diffusion sink therefore may be unlikely to diffuse out of this region in their active functional state, and thus may be effectively silenced. An illustrative example may include a hydrocephalic shunt, which is generally made of a solid walled silicone catheter with holes machined into the tip for drainage. The biocompatibility of these devices may be improved by making the entire catheter out of tubing permeable to water and molecules soluble in water, but impermeable to cells. Similarly, deep brain stimulating electrodes, which generally are solid tubular implants, may be redesigned to be permeable to water and molecules soluble in water but impermeable to cells.

The methods and apparatuses disclosure herein are not limited in their applications to the details of construction and the arrangement of components described herein. The methods and apparatuses are capable of other embodiments and of being practiced or of being carried out in various ways. Also it is to be understood that the phraseology and terminology used herein is for the purpose of description only, and should not be regarded as limiting. Ordinal indicators, such as first, second, and third, as used in the description and the claims to refer to various structures, are not meant to be construed to indicate any specific structures, or any particular order or configuration to such structures. All methods described herein can be performed in any suitable order unless otherwise indicated herein or otherwise clearly contradicted by context. The use of any and all examples, or exemplary language (e.g., “such as”) provided herein, is intended merely to better illuminate the methods and apparatuses disclosed herein and does not pose a limitation on the scope of the methods and apparatuses unless otherwise claimed. No language in the specification, and no structures shown in the drawings, should be construed as indicating that any non-claimed element is essential to the practice of the methods and apparatuses disclosed herein.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration, volume or the like range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Further, no admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein.

### EXAMPLES

### Example 1

### Modeling Studies

Various modeling studies were performed to evaluate whether diffusion sinks may be used to passively draw molecules secreted during the FBR (e.g., cytokine factors, etc.) away from tissues in which they provoke deleterious responses. The models simulated the theoretical concentrations of cytokine TNF-α, at steady state equilibrium, as a function of position relative to regions surrounding the interface between the semi-permeable exterior surface of a diffusion sink and the tissue of a host. The models also assessed optimal dimensions of various diffusion sinks for various types of medical devices.

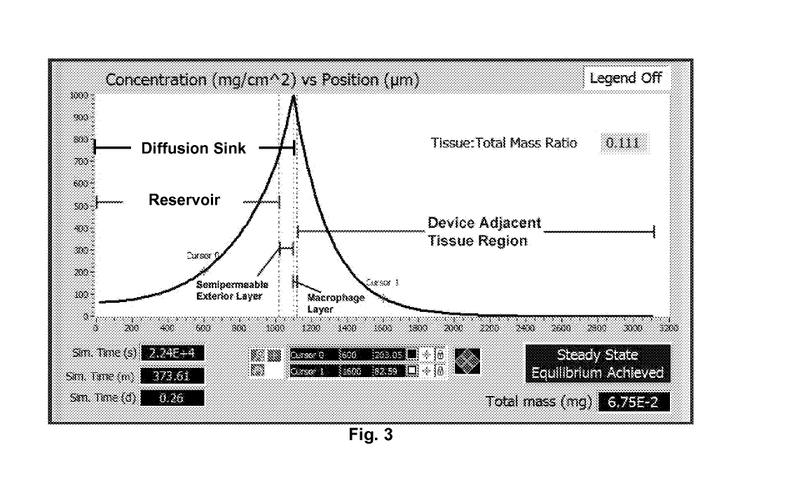

FIG. 3 is a graph showing a plot of the theoretical concentration of cytokine TNF-α, at steady state equilibrium, as a function of position relative to regions surrounding the flat interface between the semi-permeable exterior surface of a diffusion sink and a thin layer of macrophages present in the tissue of a host. Labeled cross-bars are used to delineate the extents of different regions of relevance to the model. The “Tissue:Total Mass Ratio” value (0.111) represents the fraction of macrophage released cytokine TNF-α that diffuses into the adjacent tissue region. The results from this simulation suggest that approximately 89% of the cytokine TNF-α released by surface attached macrophages will be drawn into the biofactor sink reservoir jacket region of the implantation device.

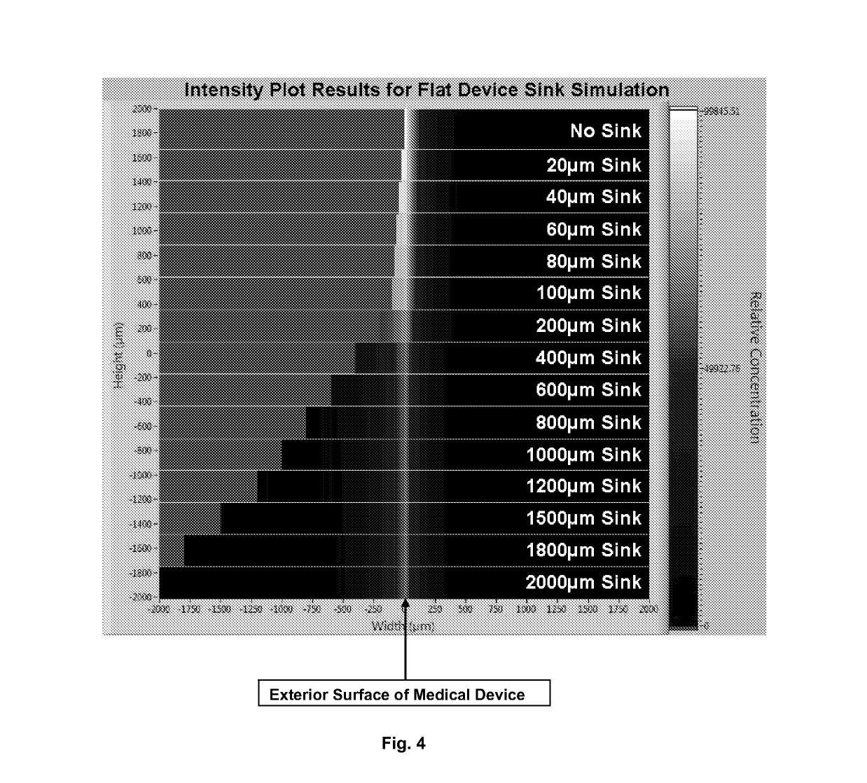

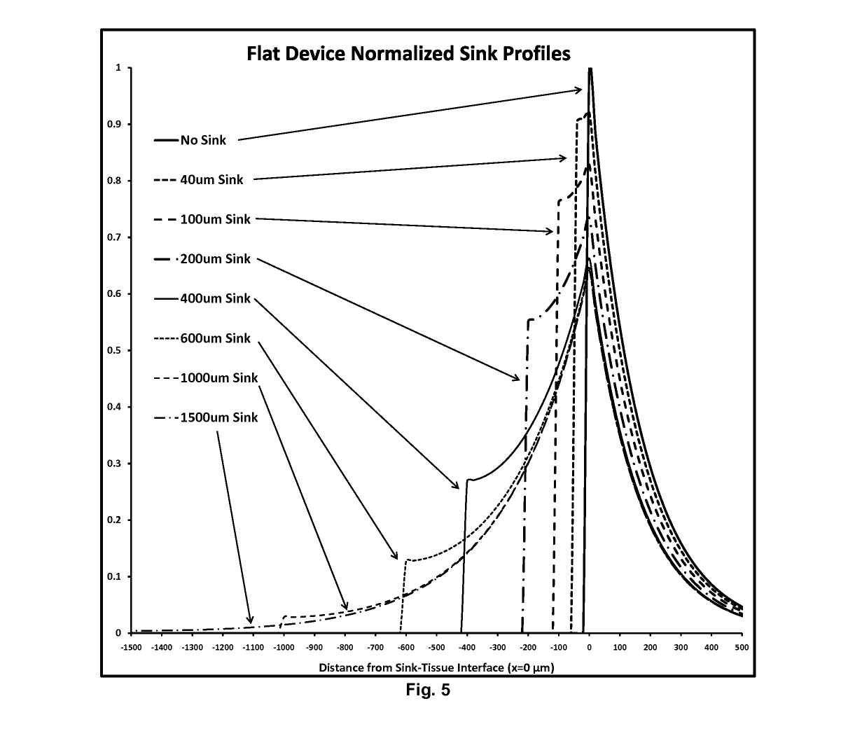

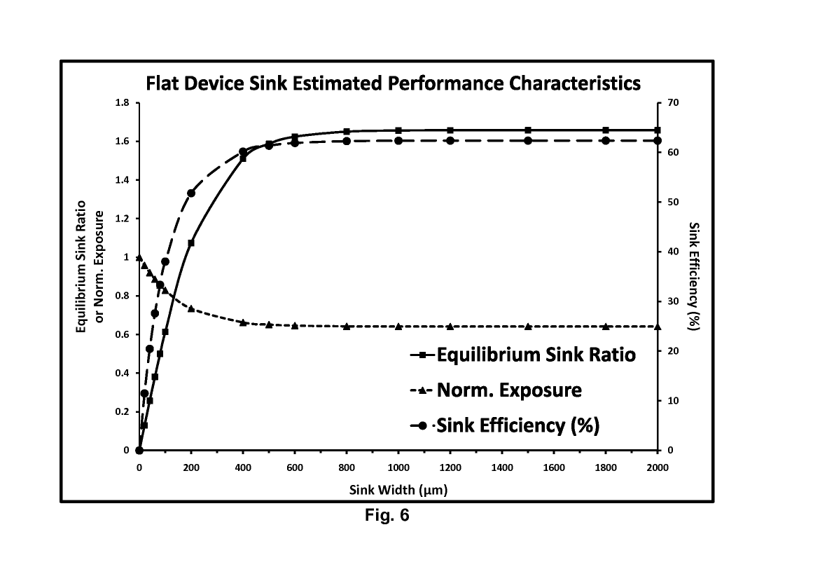

FIGS. 4-6 show the results of modeling studies predicting the theoretical concentrations of cytokine TNF-α, at steady state equilibrium, in regions surrounding the interfaces between the exterior surfaces of various flat medical devices, each having a different thickness of diffusion sink, and the tissues of hosts within which each medical device is implanted. The medical devices modeled in FIGS. 4-6 each have a flat impermeable core with a different thickness of semi-permeable diffusion sink. FIG. 4 is a schematic showing the theoretical concentrations of cytokine TNF-α, at steady state equilibrium, in regions surrounding the interfaces between the exterior surfaces of the various flat medical devices and the tissues of hosts within which each medical device is implanted. The concentration of cytokine TNF-α is represented based on the intensity of the white band surrounding the interface, as shown in the scale along the right hand side of the schematic. FIG. 5 is a graph showing plots of the theoretical concentrations of cytokine TNF-α as a function of distance from the interface between the exterior surface of the medical devices and the tissues of the host. FIG. 6 is a graph showing plots of the theoretical equilibrium sink ratio (i.e., the ratio of cytokine TNF-α in a diffusion sink to cytokine TNF-α in the surrounding tissue of the host at steady state equilibrium) and the sink efficiency (i.e., the percentage of total cytokine TNF-α in the diffusion sink at steady state equilibrium) as functions of the width of the diffusion sink for flat medical devices. As shown in FIG. 6, the optimal sink efficiency for a flat medical device is slightly greater than 60%, and this optimal efficiency is achieved at sink widths greater than about 400 μm.

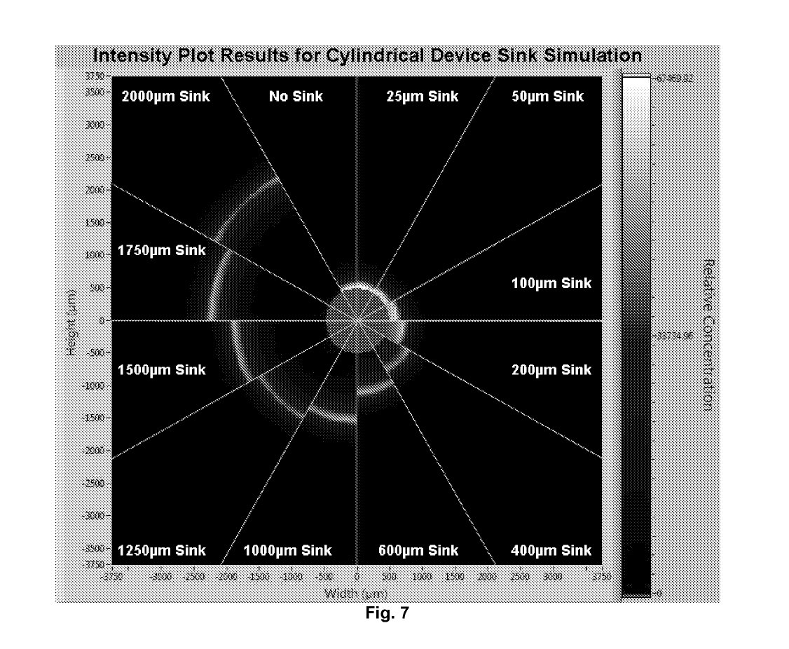

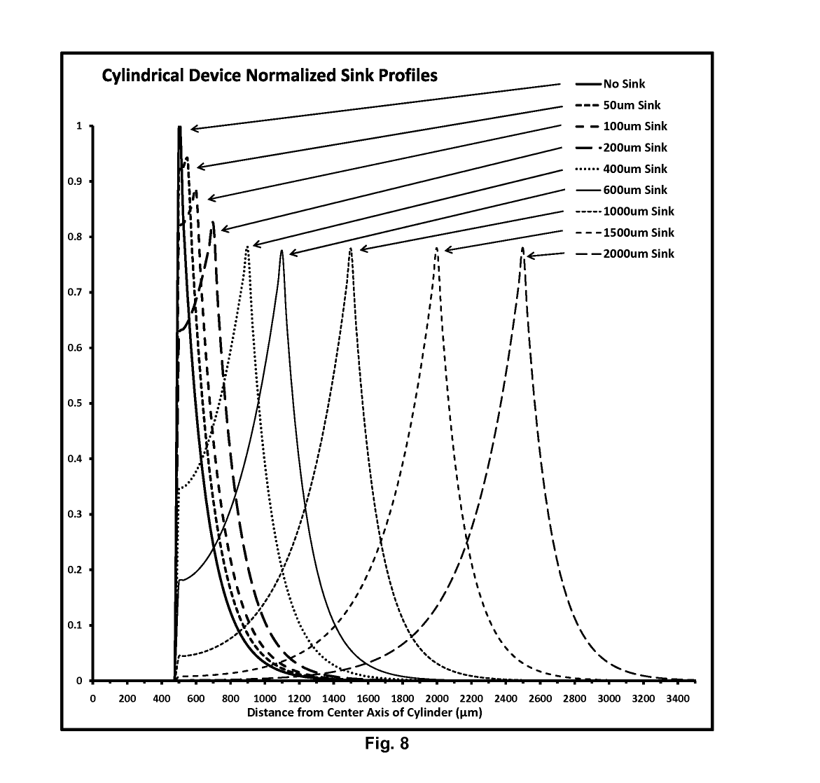

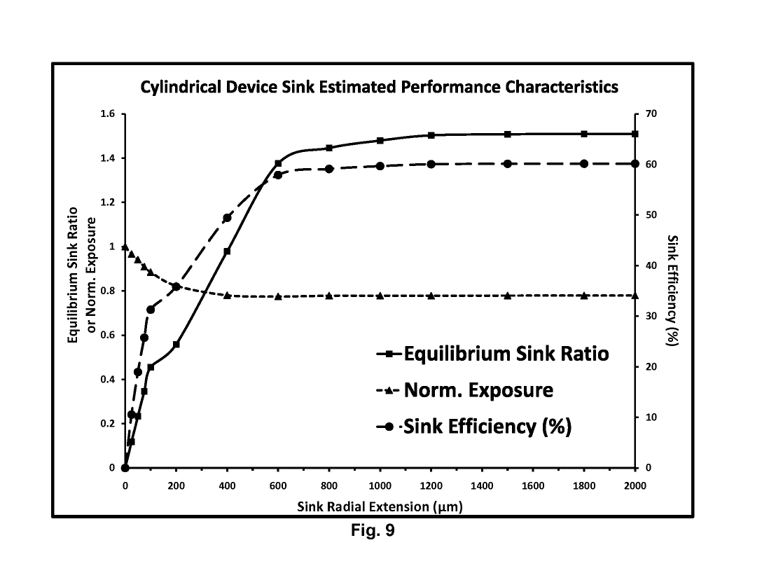

FIGS. 7-9 show the results of modeling studies predicting the theoretical concentrations of cytokine TNF-α, at steady state equilibrium, in regions surrounding the interfaces between the exterior surfaces of various cylindrical medical devices and the tissues of hosts within which each medical device is implanted. The medical devices modeled in FIGS. 7-9 each have an impermeable cylindrical core with a diameter of 1 mm and a different thickness of semi-permeable diffusion sink. FIG. 7 is a schematic showing the theoretical concentrations of cytokine TNF-α, at steady state equilibrium, in regions surrounding the interfaces between the exterior surfaces of the various cylindrical medical devices and the tissues of hosts within which each medical device is implanted. The concentration of cytokine TNF-α is represented based on the intensity of the white band surrounding the interface, as shown in the scale along the right hand side of the schematic. FIG. 8 is a graph showing plots of the theoretical concentrations of cytokine TNF-α as a function of distance from the interface between the exterior surface of the medical devices and the tissues of the host. FIG. 9 is a graph showing plots of the theoretical equilibrium sink ratio and the sink efficiency as functions of the width (i.e., the sink radial extension) of the diffusion sink for cylindrical medical devices with impermeable cylindrical cores. As shown in FIG. 9, the optimal theoretical sink efficiency for a such cylindrical medical devices is approximately 60%, and this optimal efficiency is achieved at sink widths greater than about 600 μm.

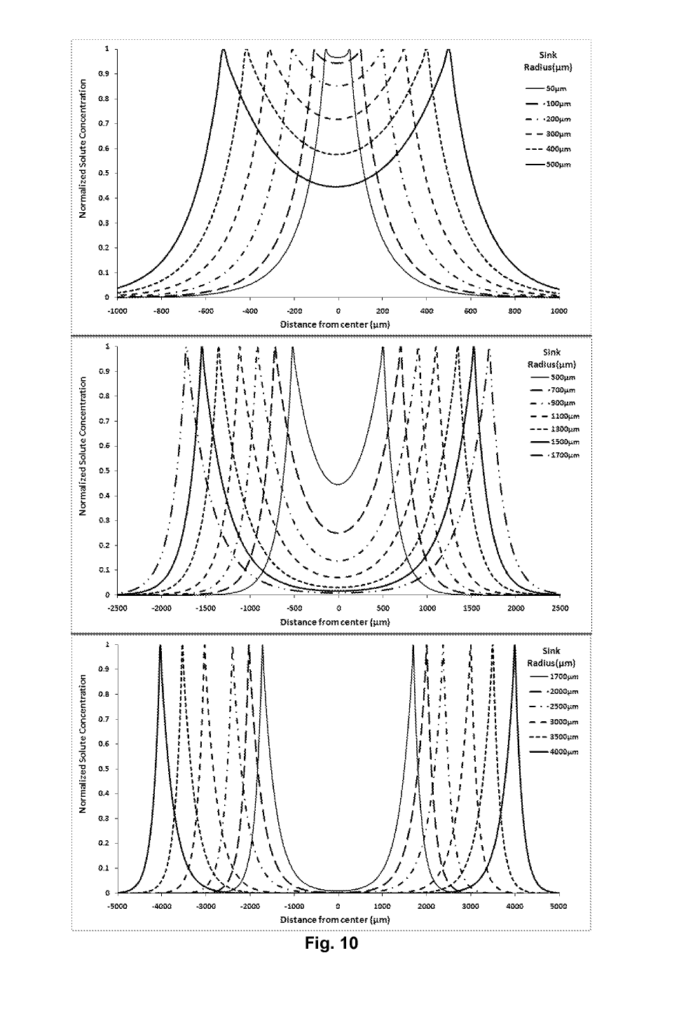

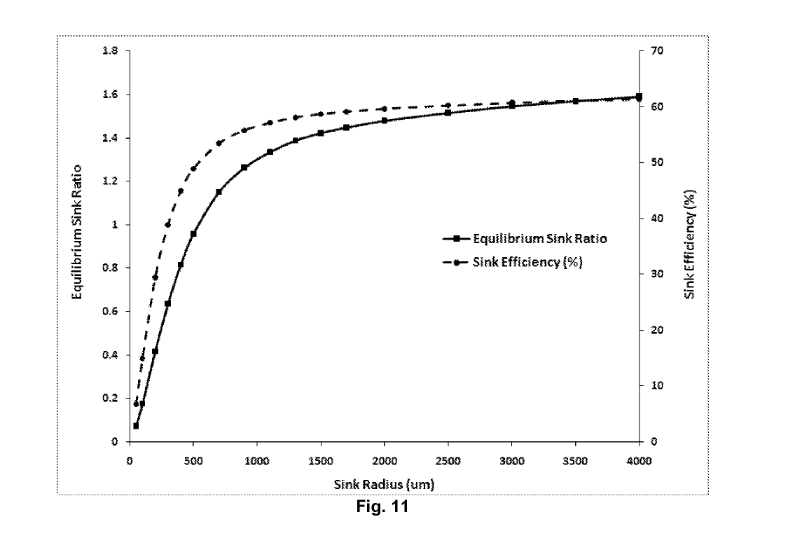

FIGS. 10-11 show the results of modeling studies predicting the theoretical concentrations of cytokine TNF-α, at steady state equilibrium, in regions surrounding the interfaces between the exterior surfaces of various cylindrical medical devices and the tissues of hosts within which each medical device is implanted. The medical devices modeled in FIGS. 10-11 do not have impermeable cores, but instead consist uniformly of semi-permeable materials having varying diameters. FIG. 10 is a series of graphs showing a series of plots of the theoretical concentrations of cytokine TNF-α as a function of distance from the interface between the exterior surface of the medical devices and the tissues of the host. FIG. 11 is a graph showing plots of the theoretical equilibrium sink ratio and the sink efficiency as functions of the width (i.e., the sink radius) of the diffusion sink for cylindrical medical devices without impermeable cylindrical cores. As shown in FIG. 11, the optimal sink efficiency for such cylindrical devices is approximately 60%, and this optimal efficiency is achieved at sink widths greater than about 600 μm.

It should be noted that any diffusion sink, regardless of its width, theoretically may improve the biocompatibility of a medical device by reducing the concentrations of cytokine TNF-α at the device-tissue interface. Even diffusion sinks with widths of about 50 μm provided for significant decreases in the theoretical amount of cytokine TNF-α in the adjacent tissues. Moreover, it should be appreciated that the overall sink efficiencies for the various devices may be improved over those shown in FIGS. 6, 9 and 11, such as by optimizing the porosity of the diffusion sinks to be as permeable to macrophage secreted molecules as possible and/or by using sink reservoirs that promote greater diffusion through the semi-permeable materials and into the reservoirs.

### Example 2

### Comparison of an Implanted Device Having an Open Reservoir to an Implanted Device Having an Occluded Reservoir

The influence of a reservoir on host tissue responses was studied by comparing the FBR of rat brain tissues to implanted devices having an open reservoir (i.e., having a reservoir) to the FBR of rat brain tissues to implanted devices having an occluded reservoir (i.e., lacking a reservoir). As discussed below, the study showed that the presence of reservoirs significantly reduces the FBR to implanted devices.

### Implantation of Devices Having Open and Occluded Reservoirs

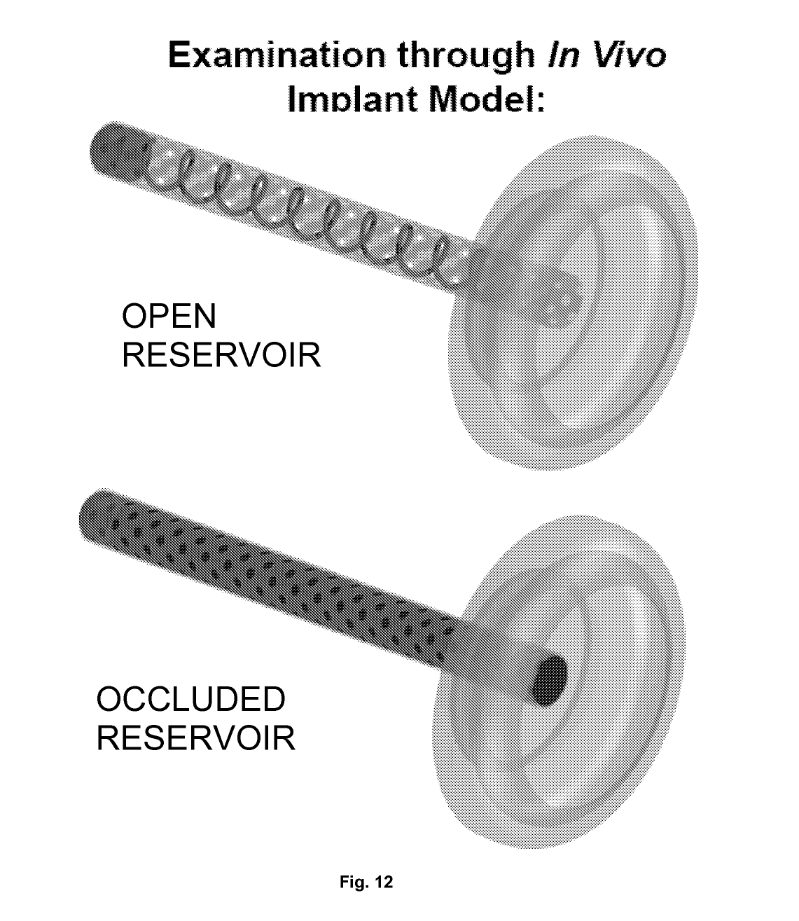

Two types of devices were implanted into rat cortical tissues: (1) devices having an open reservoir; and (2) devices having an occluded reservoir. FIG. 12 is a schematic showing cylindrical implantable devices similar to those used for the present comparison. The open reservoir device (top) consisted of a cylindrical tube formed of a semi-permeable synthetic polymer and having a hollow core. While the hollow core did have a coil within its interior, it did not contain a therapeutic device. The coil did not occlude the pores of the semi-permeable tube. This device configuration was selected for the comparison study because the reservoir could be occluded by placing a solid impermeable tubular device within the hollow core (bottom). As such, the adjacent host tissue would be exposed to a consistent exterior surface regardless of whether the reservoir of the implanted device was open or occluded. Contrasting observations for the two study groups (open or occluded) would directly correlate to the presence or absence of a sink reservoir.

Prior to implantation, the devices were pre-sterilized by exposure to UV light for 20 minutes. All surgical techniques were performed in accordance with protocols approved by the University of Utah Institutional Animal Care and Use Committee. Adult male rats were anesthetized with a mixture of ketamine, xylazine, and acepromazine, and the devices were implanted as previously described at +0.2 mm forward of bregma and 3 mm lateral to the midline. The skin incision was closed and the animals were allowed to recover and given food and water ad libitum. The rats were allowed to remain unperturbed for a 12 week indwelling period.

### Euthanasia and Isolation of Electrodes

Twelve weeks following implantation, the rats were anesthetized with a mixture of ketamine and xylazine and then transcardially perfused with ice-cold PBS, followed by 4% (w/v) paraformaldehyde in PBS. The brains were removed and the devices were retrieved with fine forceps. Specimens were post-fixed overnight in 4% paraformaldehyde prior to sectioning and immunostaining. Brains were cut to horizontal sections with a vibratome and processed for immunostaining using cell type specific markers.

### Immunostaining

Tissue sections of the cerebral cortex of each rat were analyzed for each marker. Sections were placed in blocking solution (PBS with 4% (v/v) goat serum, 0.3% (v/v) Triton X-100, and 0.1% (w/v) sodium azide) for 1 hr. Sections were then incubated in primary antisera overnight at 4 C. The following primary antibodies were used: antisera against ED-1 (Serotec) to label activated microglia and macrophages, and antisera against glial fibrillary acidic protein (GFAP; Dako) to label astrocytes. All primary antibodies were diluted 1:1000 in blocking solution. Alexa-Fluor conjugated secondary antibodies were diluted 1:200 in blocking solution and applied to the tissue sections for 1 hr at room temperature. The sections were then counterstained with 10 μm DAPI to identify all cell nuclei. The stained sections were mounted on glass slides in Fluoromount-G (Southern Biotechnology), an anti-fade reagent.

### Comparison of Tissues Around Open Reservoir Devices to Tissues Around Occluded Reservoir Devices

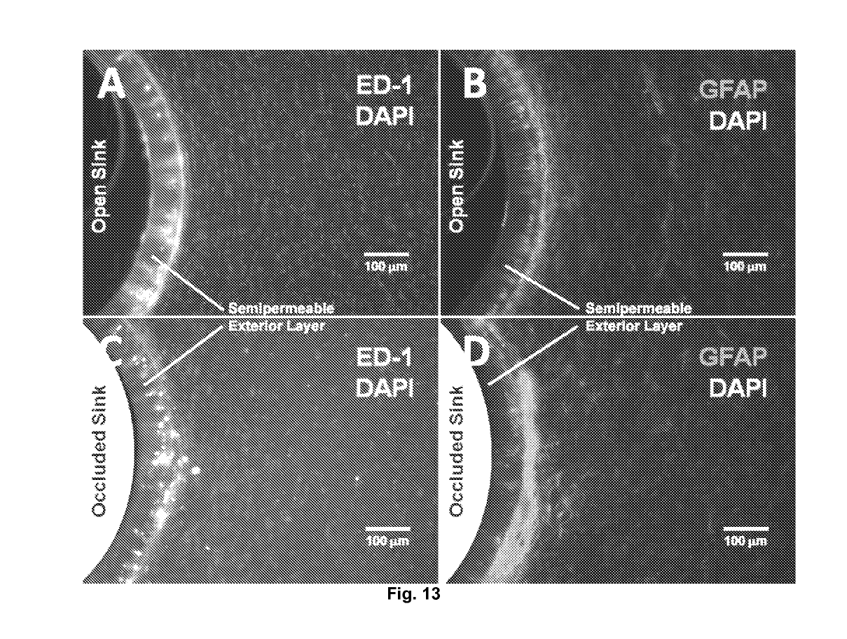

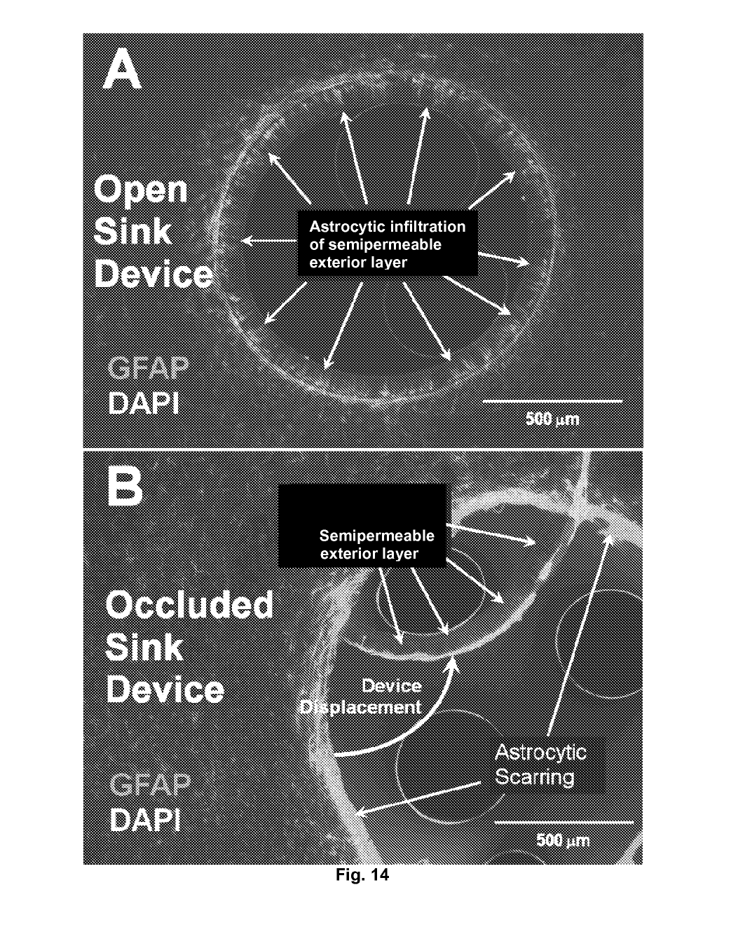

FIGS. 13 and 14 are fluorescent microscopy images of the immunostained cortical brain tissues surrounding the implantation tract of the subject rats 12 weeks after implantation. The reaction of the tissues to open and occluded reservoir devices was characterized by the presence of ED-1+ macrophages (FIGS. 13A and 13C) and GFAP+ astrocytes (FIGS. 13B, 13D, 14A and 14B) adjacent to and filling the implantation tract.

A comparison of FIGS. 13A and 13C suggests a significantly greater population of ED-1+macrophages persists at the semi-permeable exterior layer region of devices with occluded reservoirs. A comparison of FIGS. 13B and 13D suggests that the relative absence of macrophages attached to the devices having open reservoirs resulted in significantly greater GFAP+ astrocytic cell infiltration of the semi-permeable exterior layer region. The intense GFAP stained region adjacent to the occluded device (FIG. 13D) is suggestive of glial encapsulation phenomena.

As shown in FIG. 14B, the device with the occluded reservoir was (frequently) displaced during sectioning, likely due to the poor extent of bio-integration by tissues adjacent to the device. In contrast, the tissues surrounding the open reservoir devices, shown in FIG. 14A, were substantially healthier. The extensive infiltration of GFAP+ astrocytes into the semi-permeable layer allows for a pseudo-continuous, robust, and likely significantly more stable biotic-abiotic interface between the host tissue and the implantation device.

These results support the conclusion that the presence of a diffusion sink that includes a reservoir in fluid communication with adjacent host tissues significantly reduces the FBR to an implantation device, thereby improving the biocompatibility of the device. The volume of the diffusion sink in the open reservoir device was approximately 25% that of the adjacent tissue, but diffusion sinks with volumes up to about 60% that of the adjacent tissue, and perhaps even higher, may be achieved. Furthermore, even more favorable results may be obtainable with semi-permeable materials having greater porosity (provided the semi-permeable material is still impermeable to macrophage cells).

### Example 3

Comparison of an Implanted Device to an Implanted Device Coated with a Hydrogel

In vitro and in vivo studies were performed to determine the effect hydrogel coatings have on the FBR of rat brain tissues to implanted devices. As discussed below, the study supports the conclusion that hydrogel coatings significantly reduce the FBR to implanted devices.

### Applying an Alginate Coating to Microelectrodes

Single shank silicon microelectrodes were obtained from the Center for Neural Communication Technology (CNCT) at the University of Michigan. A subset of electrodes was coated with a sterile alginate hydrogel (Pronova MPG; Biopolymers Inc.; 1:1 guluronic acid:mannuronic acid). Briefly, a 1% (w/v) solution of alginate in distilled water was created under stirring conditions for 20 hrs. Single shank silicon microelectrodes were dip-coated in alginate, followed by immersion into 0.5% (w/v) calcium sulfate in distilled water. The divalent cations present in calcium sulfate initiate crosslinking of the alginate hydrogel through interactions between the units of guluronic acid. The thickness of the hydrogel coating was controlled by the number of dips into the alginate solution and was set to a nominal thickness of approximately 50 μm. Following dip coating, the alginate-coated microelectrodes were frozen at −20° C., immediately transferred to vacuum-sealed containers, and lyophilized at <10 mbar until dry.

### Isolation and Preparation of Mixed Cortical Cultures

Primary mixed cortical cultures were isolated using various methods. Cerebral cortices of P1 Sprague Dawley rats were isolated and minced after removing the meninges. The tissue was then digested with 1.33% Collagenase type IV in DMEM/F12 at 37° C. for 30 min. Next, the solution was centrifuged, the supernatant was removed by aspiration, and 0.25% trypsin and 0.02% EDTA was added for 20 min at 37° C. The resulting solution was treated for 2 min with SBTI (1.5 mg/mL soybean trypsin inhibitor) followed by centrifugation and aspiration of the supernatant. Next the cell pellet was titrated in growth media with 1% DNAse using a fire-polished Pasteur pipette, and the cell suspension was plated. Cells were allowed to grow to confluence at 37° C./5% CO2. Upon reaching confluence the cells were released using trypsin and the cell suspension was collected and centrifuged. The resulting cell pellet was resuspended in DMEM/F12 with 10% Fetal Bovine Serum (FBS), triturated with a fire-polished Pasteur pipette, and assayed for viability.

In vitro Cell Attachment Assay

Primary P1 rat-derived mixed cortical cultures were seeded at a density of 100,000 cells/ml onto sterilized uncoated and alginate-coated, 300 μm-wide Michigan-style microelectrodes attached to glass coverslips with a UV-curable adhesive (MD-1187M, Dymax, Torrington, Conn.) at the base of each electrode (n=6 per condition). Cells were cultured for 72 hrs at 37° C./5% CO2 in DMEM/F12 with 10% FBS. Following culture, cells were fixed with 4% paraformaldehyde in PBS. Cells were then stained with phalloidin (invitrogen) to label cell's actin cytoskeleton and counterstained with DAPI to identify cell nuclei. The stained electrodes and coverslips were mounted on glass slides in Fluoromount-G (Southern Biotechnology) and images were collected using a CoolSnap CCD camera attached to a Nikon E600 microscope.

### Implantation of Microelectrode Arrays

Two types of microelectrode arrays were implanted: (1) uncoated single shank microelectrodes; and (2) single shank microelectrodes with a lyophilized, alginate hydrogel coating with a thickness of 50 microns. The lyophilized hydrogel-coated microelectrodes were stored dry and implanted without pre-hydration. Prior to implantation, all electrodes were re-sterilized by exposure to UV light for 20 minutes. All surgical techniques were performed in accordance with protocols approved by the University of Utah Institutional Animal Care and Use Committee. Adult male Sprague-Dawley rats (250-274 g; Harlan) were anesthetized with a mixture of ketamine (65 mg/kg), xylazine (7.5 mg/kg), and acepromazine (0.5 mg/kg). The electrodes were implanted at +0.2 mm forward of bregma and 3 mm lateral to the midline. A subset of alginate-coated electrodes were removed immediately following implantation and grossly examined to ensure that the hydrogel coating had not sheared off. The skin incision was closed with 5-0 silk sutures and the animals were allowed to recover and given food and water ad libitum. Cohorts of at least 5 animals were implanted with alginate-coated and uncoated electrodes, respectively, and allowed to remain unperturbed for a 12 week indwelling period.

### Euthanasia and Isolation of Electrodes

Twelve weeks following implantation, animals were anesthetized with a mixture of ketamine (70 mg/kg) and xylazine (30 mg/kg) and then transcardially perfused with 125 mL of ice-cold PBS, followed by 125 mL of 4% (w/v) paraformaldehyde in PBS. The brains were removed and the electrodes were retrieved with fine forceps. The retrieved hydrogel-coated electrodes were examined microscopically to determine if the alginate coating was still present. Both specimens were post-fixed overnight in 4% paraformaldehyde prior to sectioning and immunostaining. Brains were cut to 40 μm horizontal sections with a vibratome and processed for immunostaining using cell type specific markers.

### Immunostaining

Six to eight tissue sections of the cerebral cortex of each animal were analyzed for each marker. Sections were placed in blocking solution (PBS with 4% (v/v) goat serum, 0.3% (v/v) Triton X-100, and 0.1% (w/v) sodium azide) for 1 hr. Sections were then incubated in primary antisera overnight at 4 C. The following primary antibodies were used: antisera against glial fibrillary acidic protein (GFAP; Dako) to label astrocytes, antisera against CD68/ED1 (Serotec) to label activated microglia and macrophages, antisera against neurofilament 160 (NF; Sigma) to label neuronal processes, and antisera against neuronal nuclei to label mature neurons (NeuN; Chemicon). All primary antibodies were diluted 1:1000 in blocking solution. Alexa-Fluor conjugated secondary antibodies were diluted 1:200 in blocking solution and applied to the tissue sections for 1 hr at room temperature. The sections were then counterstained with 10 μm DAPI to identify all cell nuclei. The stained sections were mounted on glass slides in Fluoromount-G (Southern Biotechnology), an anti-fade reagent. Retrieved electrodes were stained in an analogous manner.

### Statistical Analysis

In order to determine differences in immunoreactivity between the alginate-coated and uncoated electrodes at 12 weeks, quantitative pixel intensity values for GFAP, CD68, and NF were integrated across discrete 50 μm wide bins and compared between the two conditions using a two-tailed t-test. The mean number of neuronal nuclei was also compared across both 50 and 100 μm wide bins, respectively, between the uncoated and coated electrodes using a two-tailed t-test. A Spearman's rank coefficient was calculated to determine the strength of correlation between GFAP, CD68, and NF reactivity, respectively. Significant differences were reported at p<0.05 for all statistical tests.

In vitro Comparison of Cortical Cell Attachment to Alginate-Coated and Uncoated Electrodes

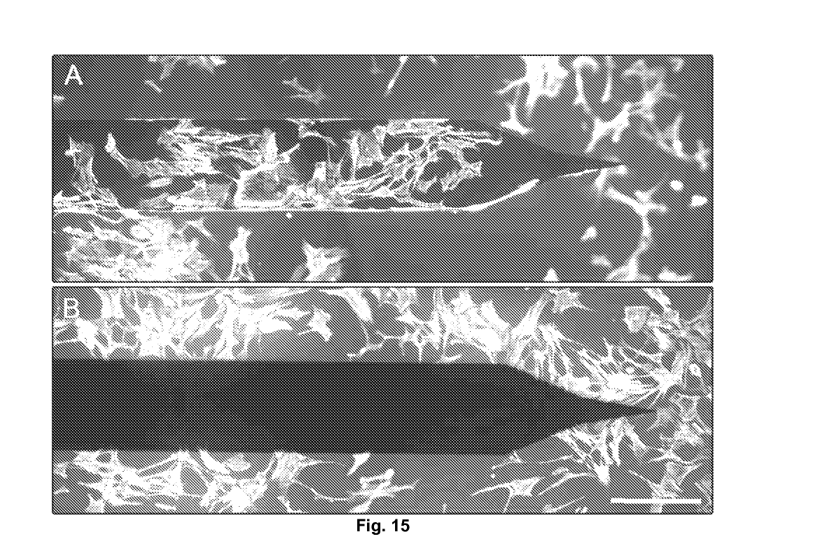

As shown in FIG. 15, cortically derived mixed cell cultures attached readily to uncoated planar silicon microelectrode arrays (FIG. 15A) but not to the same microlelectrode arrays coated with alginate (FIG. 15B).

### Alginate-Coated and Uncoated Electrodes

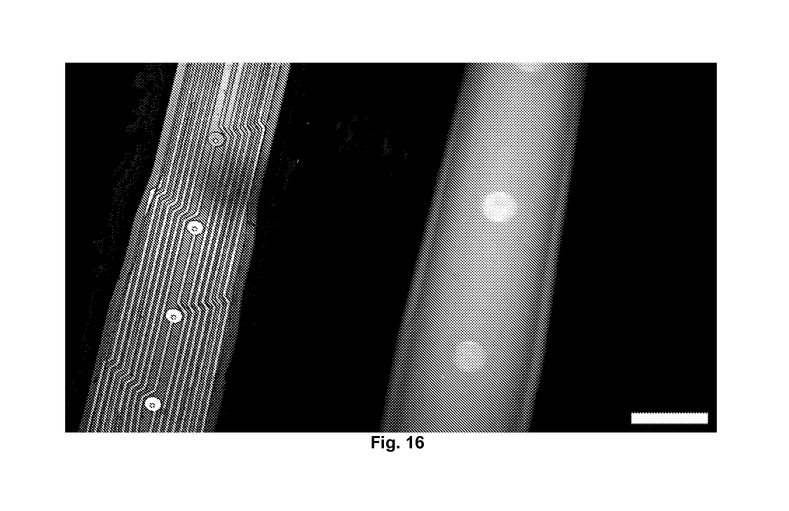

FIG. 16 shows two electrodes upon hydration in vitro to illustrate the appearance of the alginate coating. Upon implant retrieval after 12 weeks in rat cortex, the alginate hydrogel coating appeared to be present in most cases upon microscopic examination of the explanted electrodes (data not shown). However, evidence of fragmentation and discontinuities in segments of the coating was observed in a subset of the retrieved electrodes. Initial examination of alginate-coated electrodes that were retrieved immediately following hydration in situ demonstrated intact hydrogel coatings, suggesting that it was not sheared off during the implantation procedure. The observed defects of retrieved electrodes may have resulted changes in the integrity of the coating following prolonged exposure to the CNS milieu which has a lower divalent ion content than that used to stabilize the coating.

Cell attachment to the surface of the retrieved microelectrodes was also analyzed. Activated macrophages (CD68+ cells) were observed on the uncoated electrodes; in contrast, minimal attachment of activated macrophages (CD68+ cells) was observed on alginate-coated microelectrodes (data not shown).

### Inflammation and Reactive Astrogliosis Around Uncoated and Alginate-Coated Electrodes

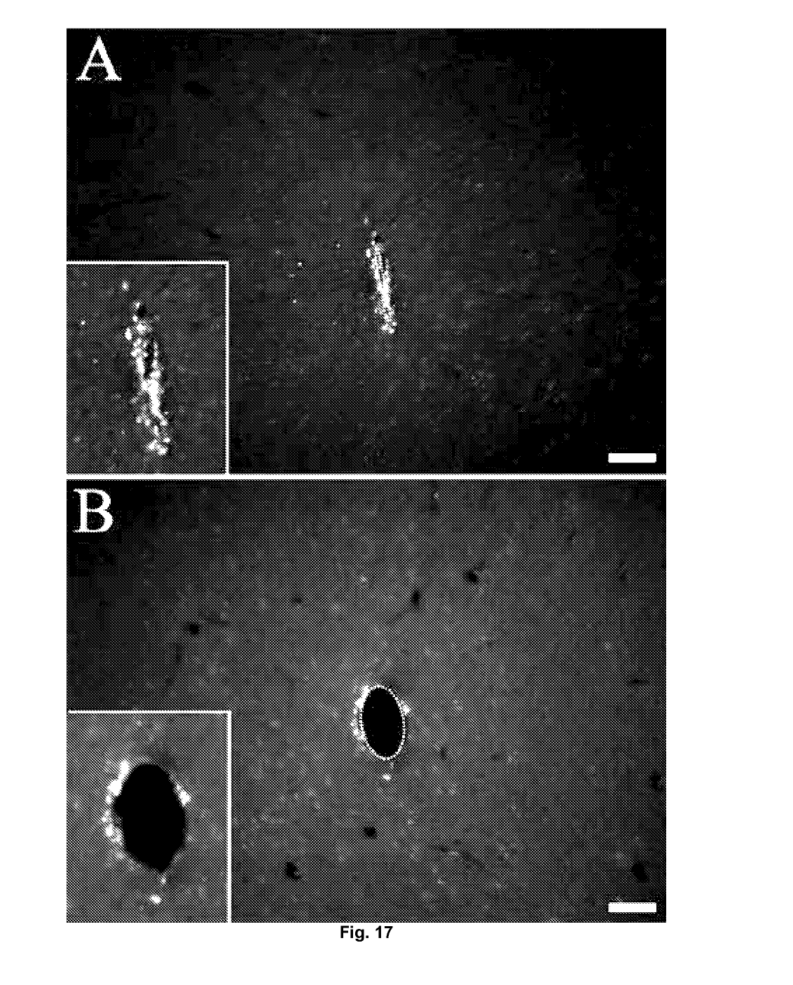

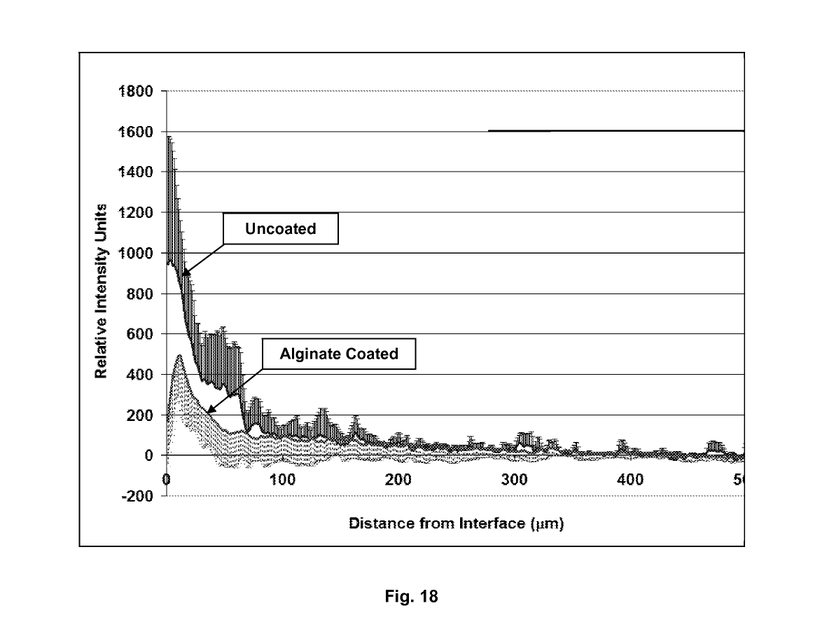

The cortical brain tissue reaction to uncoated microelectrodes was characterized by the presence of CD68+ macrophages adjacent to and filling the implantation tract (FIG. 17A). In contrast, for alginate-coated microelectrodes, the distribution of CD68+ immunoreactivity was reduced, with sporadic clusters of CD68+ immunoreactivity asymmetrically distributed around the biotic-abiotic interface of (FIG. 17B). As shown in FIG. 18, quantitative analysis of the spatial distribution and relative intensity of the response showed that the response was significantly reduced for the alginate-coated electrode cohort, characterized by a lower peak of CD68 intensity approximately 11 μm away from the interface.

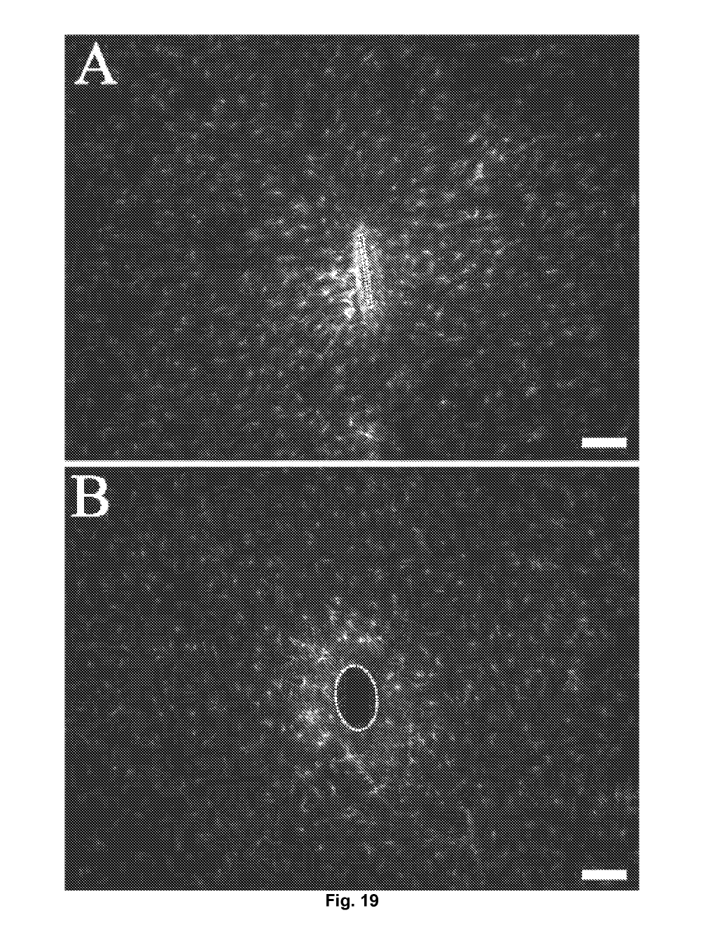

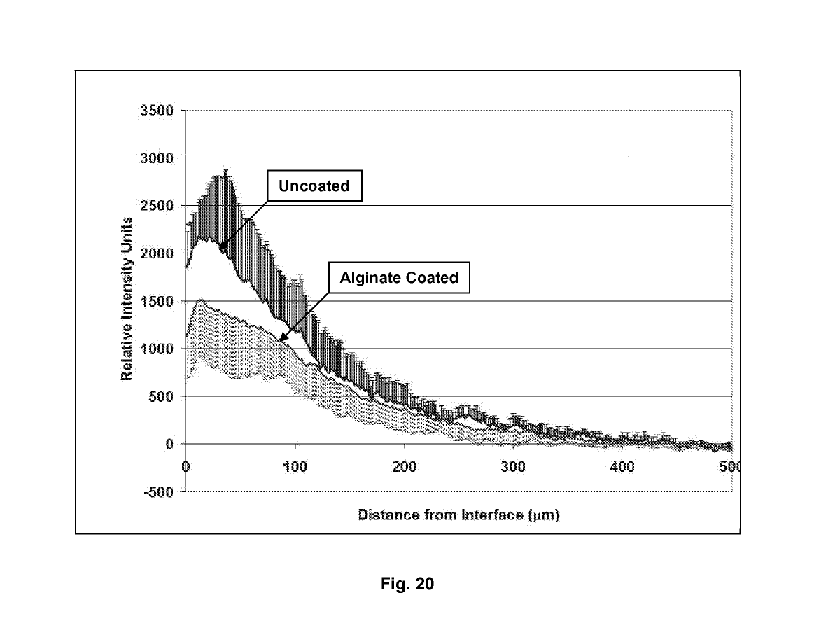

A region of hypertrophic GFAP+ astrocytes was observed surrounding the CD68+immunoreactive zone in both groups; however, the spatial arrangement of the marker was quite different. For tissues surrounding the uncoated electrodes, we observed a hypertrophic astrocytic response around the implantation tract, forming a zone of tissue that spanned approximately 50 μm in width (FIG. 19A). In contrast, the spatial arrangement of hypertrophic astrocytes around the hydrogel coated group was different (FIG. 19B), and instead was similar to that of uninjured tissue, with the exception of a few sporadic hypertrophic astrocytes observed scattered at the area of the biotic-abiotic interface. In some subjects, a zone devoid of GFAP+ cells was observed between the microelectrode-tissue interface and the surrounding layer of GFAP+ astrocytes. As shown in FIG. 20, quantitative analysis of the GFAP response showed that the response was less in the alginate-coated electrode cohort.

### Reduction in Neuronal Density Around Uncoated and Alginate-Coated Electrodes

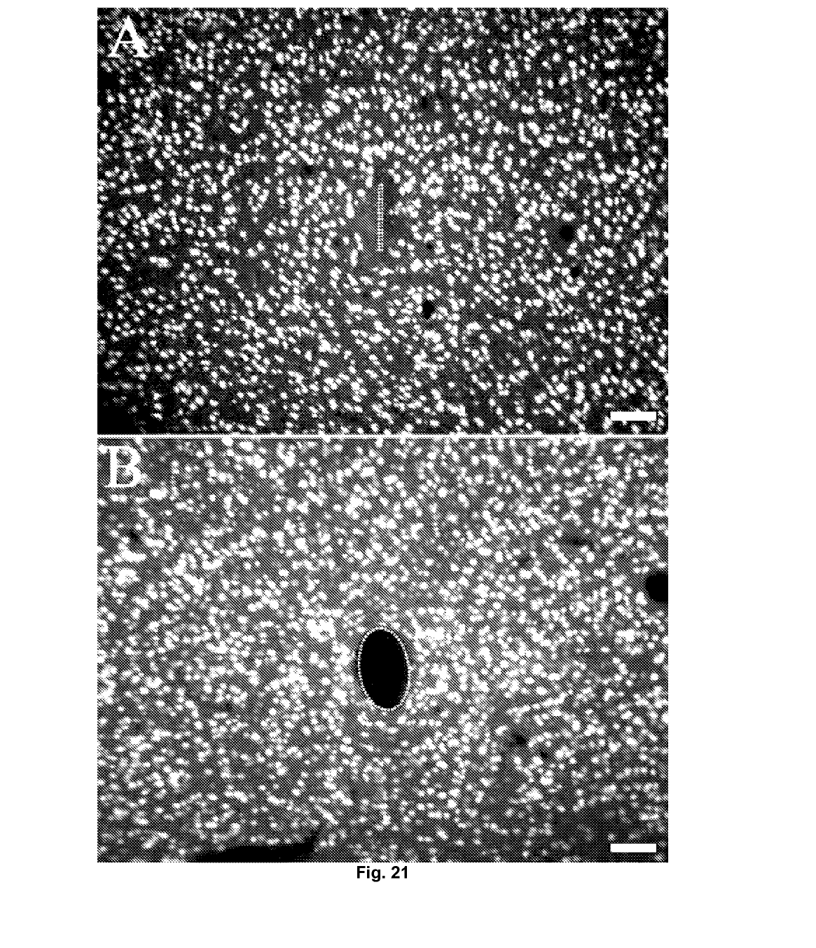

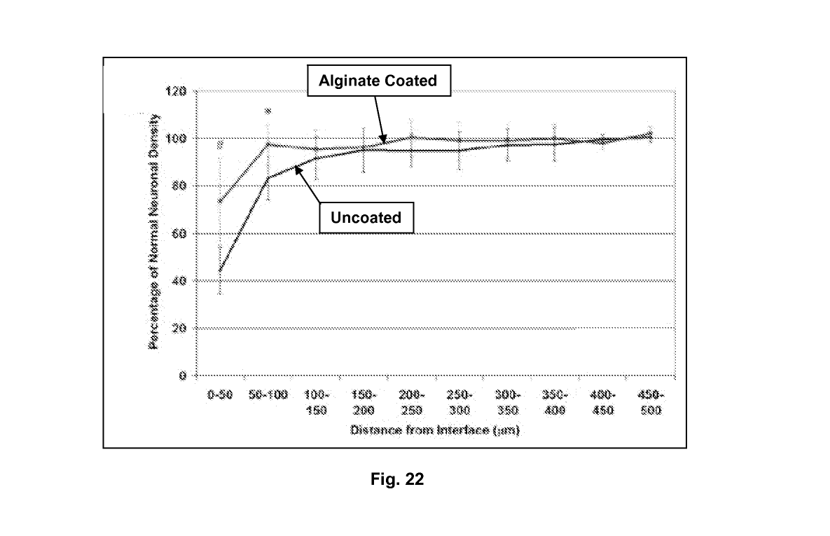

A reduction in the density of NeuN+ neuron nuclei was also observed within a 100 μm radius from the uncoated electrode-tissue interface (FIG. 21A). An analysis of neuronal density in 50 μm wide bins demonstrated that much of the reduction in neuronal density occurred within the first 50 μm directly adjacent to the biotic-abiotic interface (FIG. 22). A reduction in neuronal density to approximately 45% of the levels found in normal tissue (from ˜7.5 to ˜3.5 NeuN+ nuclei per 0.005 mm2) was observed in this region adjacent to uncoated indwelling microelectrodes. As shown in FIG. 21B and 22, the number of NeuN+ nuclei was significantly higher in this region adjacent to alginate-coated microelectrodes (p<0.01), as the density was only reduced to 73% of normal levels (˜5.5 NeuN+ nuclei per 0.005 mm2). Of particular interest, the neuronal nuclei density was close to normal levels (˜97% of normal) in the 50 to 100 μm region adjacent to the biotic-abiotic interface for alginate-coated probes, and was significantly higher than the density of neuronal nuclei in this area surrounding uncoated electrodes (p<0.05).

### Area-Under-the-Curve (AUC) Analysis of the Brain Tissue Response to Uncoated and Alginate-Coated Electrodes

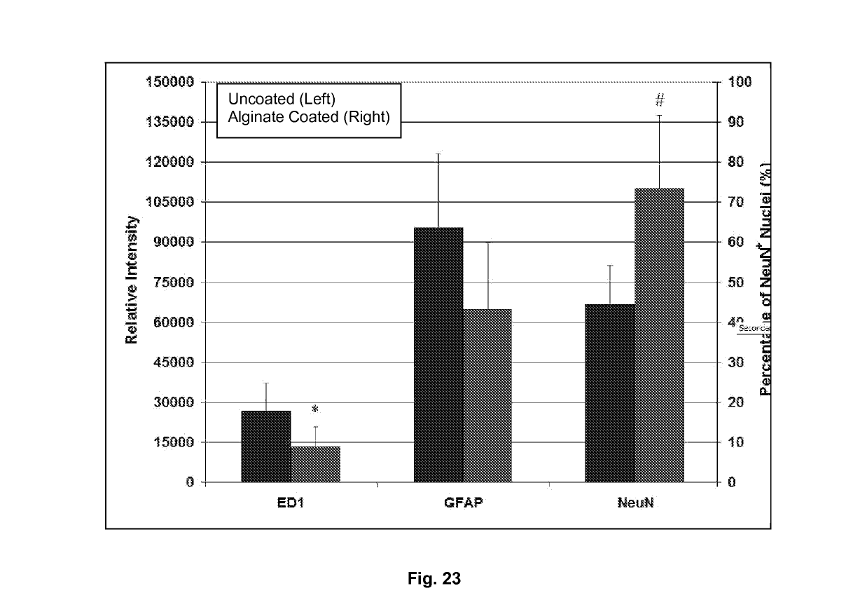

Differences in the cellular response between uncoated and alginate-coated electrodes was also analyzed using an area-under-the-curve (AUC) analysis of the respective biomarker intensity distributions. Mean pixel intensity for each immunomarker was integrated across discrete 50 μm wide bins from the electrode-tissue interface out to 500 μm away. The integrated AUC in the 50 μm closest to the electrode-tissue interface was compared with that of “normal tissue” for GFAP, CD68, and NF, respectively, and found to be significantly different for each cell marker (data not shown). Comparison of the integrated AUC between the uncoated and alginate-coated probes demonstrated significant differences in CD68 reactivity (p<0.05) and NeuN+ neuronal nuclei density (p<0.01) within 50 μm from the interface (FIG. 23). While a trend towards reduced astrogliosis adjacent to alginate-coated electrodes was also observed in this region, there was no significant difference in the AUC measurement for GFAP between alginate-coated and uncoated electrodes (p=0.09).

Correlation between CD68 and NF reactivity for uncoated and alginate-coated electrodes.

Correlation coefficients were calculated between CD68 and NF immunoreactivity at 12 weeks post-implantation. A strong inverse correlation was observed between CD68 and NF immunoreactivity for both the uncoated electrodes (Spearman coefficient of −0.975; p<0.01) and the alginate-coated electrodes; however, the correlation was not as strong for the alginate-coated electrodes (Spearman coefficient of −0.830; p<0.01). As discussed earlier, a shift in the peak intensity away from the electrode-tissue interface was observed for CD68 in the alginate-coated microelectrodes. This shift may account for the decreased Spearman coefficient observed for alginate-coated electrodes. Further analysis indicated that there was no correlation between GFAP and NF immunoreactivity or GFAP and ED1 immunoreactivity, respectively.

1. An implantable medical device configured to be at least partially implanted within a tissue of a host, comprising:
an outer surface at least a portion of which is impermeable to cells but is permeable to water and molecules secreted by cells, the outer surface separating a space inside the medical device from a surrounding tissue of the host; and
a diffusion sink positioned within the space and configured to cause molecules that are secreted into the tissue by cells during a foreign body response (FBR) to diffuse through the portion and into the diffusion sink.

2. The implantable medical device of claim 1, wherein the diffusion sink includes a semi-permeable material, and the portion of the outer surface includes an outer surface of the semi-permeable material.

3. The medical device of claim 1, wherein the medical device includes a class II or class III medical device having an outer wall at least partially formed of the semi-permeable material.

4. The medical device of claim 1, wherein the medical device includes a class II or class III medical device having an outer wall formed of a substantially impermeable material, and wherein the semi-permeable material has been applied to at least a portion of the outer wall of the class II or class III medical device to form the medical device.

5. The medical device of claim 1, wherein the semi-permeable material includes at least one of a hydrogel, a porous film, or a membrane.

6. The medical device of claim 1, wherein the semi-permeable material includes a layer that is at least about 50 μm thick.

7. The medical device of claim 6, wherein the layer is at least about 400 μm thick.

8. The medical device of claim 6, wherein the layer is at least about 600 μm thick.

9. The medical device of claim 1, wherein the semi-permeable material includes a plurality of pores sized to accommodate the passage of molecules and to preclude passage of cells.

10. The medical device of claim 1, wherein the diffusion sink includes a reservoir that is separated from the tissue by the semi-permeable material, and is in fluid communication with the portion of the outer surface due to the permeability of the semi-permeable material to water.

11. The medical device of claim 10, wherein the reservoir is at least partially defined by at least one wall formed of an impermeable material.

12. The medical device of claim 10, wherein the reservoir is at least partially filled with the semi-permeable material.

13. The medical device of claim 10, wherein the semi-permeable material is a first semi-permeable material having a first plurality of pores having a first average pore size, and the reservoir is at least partially filled with a second semi-permeable material having a second plurality of pores having a second average pore size that is larger than the first average pore size.

14. The medical device of claim 1, wherein the medical device includes a plurality of surface members, each surface member extending outwardly to an end, wherein the semi-permeable material forms a layer that engages the ends of the surface members.

15. The medical device of claim 1, wherein the volume of the diffusion sink is at least about 25% the volume of the adjacent tissue.

16. A method of improving the biocompatibility of a medical device, comprising:
providing a class II or class III medical device having an outer wall;
applying a semi-permeable material to at least a portion of the outer wall to form a diffusion sink having an outer surface that is impermeable to cells but is permeable to molecules secreted by cells, wherein the diffusion sink is configured to cause molecules that are secreted by cells adjacent to the outer surface during a foreign body response (FBR) to diffuse through the portion and into the diffusion sink.

17. The method of claim 16, wherein the outer wall of the class II or class III medical device is substantially impermeable to water.

18. The method of claim 16, wherein the semi-permeable material includes at least one of a hydrogel, a porous film, a membrane or a porous media that permits the diffusive transport of molecules into its bulk dimensions.

19. The method of claim 16, wherein the semi-permeable material is applied to a thickness of at least about 50 μm.

20. The method of claim 19, wherein the semi-permeable material is applied to a thickness of at least about 400 μm thick.

21. The method of claim 19, wherein the semi-permeable material is applied to a thickness of at least about 600 μm thick.

22. The method of claim 16, wherein the diffusion sink includes a reservoir that is in fluid communication with the outer surface due to the permeability of the semi-permeable material to water.

23. The method of claim 22 wherein the reservoir is at least partially defined by at least one wall formed of an impermeable material.

24. The method of claim 22, wherein the reservoir is at least partially filled with the semi-permeable material.

25. The method of claim 16, wherein the class II or class III medical device includes a plurality of surface members, each surface member extending outwardly to an end, wherein the semi-permeable material forms a layer that engages the ends of the surface members.
